SEQUENCE LISTING (5 sequences)

SEQ ID NO: 1 - protein, 107 aa, Artificial Sequence
  Synthetic Construct
DIQMTQSPSSLSASVGDRVTITCRASKDISKYLNWYQQKPGKAPKLLIYYTSGYHSGVPSRFSGSGSGTDFTLTISSLQPEDFATYYCQQGDALPPTFGGGTKVEIK

SEQ ID NO: 2 - protein, 119 aa, Artificial Sequence
  Synthetic Construct
QVQLVQSGAEVKKPGSSVKVSCKASGYTFGNYWMQWVRQAPGQGLEWMGAIYEGTGKTVYIQKFADRVTITADKSTSTAYMELSSLRSEDTAVYYCARLSDYVSGFGYWGQGTTVTVSS

SEQ ID NO: 3 - protein, 214 aa, Artificial Sequence
  Synthetic Construct
DIQMTQSPSSLSASVGDRVTITCRASKDISKYLNWYQQKPGKAPKLLIYYTSGYHSGVPSRFSGSGSGTDFTLTISSLQPEDFATYYCQQGDALPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 445 aa, Artificial Sequence
  Synthetic Construct
QVQLVQSGAEVKKPGSSVKVSCKASGYTFGNYWMQWVRQAPGQGLEWMGAIYEGTGKTVYIQKFADRVTITADKSTSTAYMELSSLRSEDTAVYYCARLSDYVSGFGYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 5 - protein, 37 aa, Homo sapiens
ACDTATCVTHRLAGLLSRSGGVVKNNFVPTNVGSKAF

(19) United States
(12) Patent Application Publication
(10) Pub. No.: US 2021/0340230 A1
(43) Pub. Date: Nov. 4, 2021

(54) Anti-CGRP Antibodies For Treatment-Resistant Patients

(71) Applicant: Eli Lilly and Company, Indianapolis, IN (US)

(72) Inventor: Sheena Kaur Aurora, Zionsville, IN (US)

(21) Appl. No.: 17/269,365

(22) PCT Filed: Aug. 21, 2019
(86) PCT No.: PCT/US2019/047502

§ 371 Date: Feb. 18, 2021

Related U.S. Application Data
(60) Provisional application No. 62/721,258, filed on Aug. 22, 2018.

(51) Int. Cl. C07K 16/18

(52) U.S. Cl. CPC C07K 16/18; A61K 2039/545; C07K 2317/76; C07K 2317/24

(57) ABSTRACT

The invention provides for methods, treatments, and uses of anti-human calcitonin gene related peptide (CGRP) antibodies for the prevention of migraine headaches in patients that have previously failed to respond to migraine preventive medications either because of inadequate efficacy, a lack of safety, and/or tolerability.

The present invention is in the field of medicine. More specifically, the present invention relates to methods and uses of antibodies directed to calcitonin gene-related peptide (CGRP) for the prevention of migraine headaches in human patients that have previously failed to respond to migraine preventive medications because of inadequate efficacy, a lack of safety, and/or tolerability.

Migraine is a common neurologic disease that affects more than 36 million people in the Unites States, and is one of the top 10 causes of disability worldwide. Despite the availability of preventive medications for migraine, many patients do not respond to these treatments or are unable to tolerate them. In countries like the United States, Germany, France, and Japan, approximately 43% of patients have experienced a failure of their preventive medication or have switched treatments. Among patients with episodic or chronic migraine who are undergoing oral preventative treatment, side effects and a lack of efficacy are the most common reasons for discontinuing their treatment. Thus, there exists a need for new treatment options for patients who suffer from migraine headaches and have previously failed migraine preventative medications. As with all therapeutic treatments, it is critical that any new treatment is not associated with an unacceptable safety and/or toxicity profile.

Accordingly, the present invention provides a surprisingly safe and effective therapeutic treatment for the prevention of migraine in a patient with treatment-resistant migraine headaches comprising administering a therapeutically effective amount of an anti-human CGRP antibody to the patient.

Antibodies directed to CGRP and methods of making the same are well known and have been previously described, for example, in International Patent Application published under the Patent Cooperation Treaty (PCT) Publication Number WO2011/156324A1. “CGRP” or “calcitonin gene related peptide” or “calcitonin gene-related peptide” refers to a peptide having the sequence given in SEQ ID NO: 5. Non-limiting examples of anti-human CGRP antibodies include eptinezumab, fremanezumab, and galcanezumab.

In some embodiments of the present invention, the anti-human CGRP antibody to be administered to the patient comprises a heavy chain having a heavy chain variable region having the amino acid sequence of SEQ ID NO: 2 and a light chain having a light chain variable region having the amino acid sequence of SEQ ID NO: 1. In some embodiments, the anti-human CGRP antibody to be administered to the patient comprises a heavy chain having the amino acid sequence of SEQ ID NO: 4 and a light chain having the amino acid sequence of SEQ ID NO: 3. In some embodiments, the anti-human CGRP antibody to be administered to the patient binds to an epitope on human CGRP comprising amino acid residues 8-18 (i.e., VTHRLAGLLSR) of human CGRP (as shown in SEQ ID NO: 5), as determined by hydrogen-deuterium exchange. The anti-human CGRP antibodies to be administered to the patient as disclosed herein are preferably formulated for parenteral administration, more preferably for subcutaneous delivery.

In general, the classes of medications commonly used to prevent migraine headaches are beta-blockers (e.g. propranolol, atenolol, metoprolol, nadolol, and timolol), calcium channel blockers (e.g., verapamil, diltiazem, nimodipine), tricyclic anti-depressants (e.g., amitriptyline, nortriptyline, imipramine), selective serotonin reuptake inhibitors (e.g., fluoxetine, paroxetine, and sertraline), anticonvulsants (e.g., divalproex sodium, gabapentin, and topiramate), serotonin antagonists (e.g., methysergide and methylergonovine), and other treatments that include magnesium salts (e.g., magnesium oxide, magnesium chloride slow release, and magnesium diglycinate), vitamins (e.g., riboflavin), and herbals (e.g., Mig-99 and petasites).

A “migraine headache” as used herein refers to headache, with or without aura, of ≥30 minutes duration, with both of the following required features (A and B): A) at least 2 of the following headache characteristics: 1) unilateral location, 2) pulsating quality, 3) moderate or severe pain intensity, and 4) aggravation by or causing avoidance of routine physical activity; and B) during headache at least one of the following: a) nausea and/or vomiting, and/or b) photophobia and phonophobia.

A “probable migraine headache” as used herein refers to a headache of ≥30 minutes duration, with or without aura, but missing one of the migraine features in the International Headache Society ICHD-3 definition.

A “migraine headache day” as used herein refers to a calendar day on which a migraine headache or probable migraine headache occurs. An “ICHD migraine headache day” as used herein refers to a calendar day on which a migraine headache occurs. A “migraine headache attack” refers to the beginning on any day a migraine headache or probable migraine headache is recorded and ends when a migraine-free day occurs. A “non-migraine headache” refers to all headaches of ≥30 minutes duration not fulfilling the definition of migraine or probable migraine. A “non-migraine headache day” refers to a calendar day on which a non-migraine headache occurs. A “headache day” refers to a calendar day on which any type of headache occurs (including migraine, probable migraine, and non-migraine headache).

“Episodic migraine” as used herein refers to 4 to 14 migraine headache days and <15 headache days per 30-day period in the prospective baseline period. “Chronic migraine” as used herein refers to at least 15 headache days per 30-day period in the prospective baseline period, of which at least 8 are migraine. A “migraine headache day” refers to a calendar day on which a migraine headache or probable migraine headache occurs.

In some embodiments, a patient is a human who has been diagnosed as having a condition or disorder in need of treatment with an antibody or pharmaceutical composition described herein. In some embodiments, a patient is a human that is characterized as being at risk of a condition or disorder for which treatment or administration with a pharmaceutical composition described herein is indicated.

As used herein, the term “treating” (or “treat” or “treatment”) refers to processes involving a slowing, interrupting, arresting, controlling, stopping, reducing, or reversing the progression or severity of a symptom, disorder, condition, or disease, but does not necessarily involve a total elimination of all disease-related symptoms, conditions, or disorders associated with CGRP activity. As used herein, the term “prevention” (or “prevent” or “preventing”) refers to precluding, averting, obviating, forestalling, reducing the incidence of, stopping, or hindering the symptoms of a disease, disorder and/or condition. Prevention includes administration to a subject who does not exhibit symptoms of a disease, disorder, and/or condition at the time of administration.

As used herein, the term “therapeutically effective amount” refers to the amount or dose of an anti-human CGRP antibody in a pharmaceutical composition, which upon single or multiple dose administration to the patient, provides the desired pharmacological effect in the patient. A dose can include a higher initial loading dose, followed by a lower dose. A “dose” refers to a predetermined quantity of a therapeutic drug calculated to produce the desired therapeutic effect in a patient.

As used herein, the terms “month,” “monthly,” and derivations thereof, refer to a time period that is from 28 to 31 consecutive days unless otherwise stated. The term “about” as used herein, means in reasonable vicinity of the stated numerical value, such as plus or minus 10% of the stated numerical value.

Non-limiting examples of propranolol include propranolol hydrochloride, ANAPRILIN®, AVLOCARDYL®, INDERAL®, OBZIDAN®, REXIGEN®, BETADREN®, DEXPROPRANOLOL®, and DOCITON®. Non-limiting examples of metoprolol include metoprolol fumarate, metoprolol succinate, metoprolol tartrate, LOPRESSOR®, BETALOC®, TORPOL®, SELOKEN®, SPESIKOR®, SPESICOR®, and TOPROL XL®. Non-limiting examples of topiramate include topiramate calcium, topiramate potassium, topiramate sodium, and TOPAMAX®. Non-limiting examples of valproate include valproate sodium, divalproex sodium, divalproex, valproic acid, DEPACON®, DEPAKENE®, and DEPAKOTE®. Non-limiting examples of amitriptyline include amitriptyline hydrochloride, amitriptyline pamoate, ELAVIL®, and LEVATE®. Non-limiting examples of flunarizine include flunarizine dihydrochloride, flunarizine hydrochloride, SIBELIUM®, FLUFENAL®, FLUVERT®, ZINASEN®, ISSIUM®, VERTIX®, NOVO-FLUNARIZINE®, and APO-FLUNARIZINE®. Non-limiting examples of candesartan include candesartan cilexetil, BIOPRESS®, ATACAND®, AMIAS®, and RATACAND®. Non-limiting examples of botulinum toxin A include onabotulinumtoxinA, BOTOX®, DYSPORT®, and XEOMIN®. Non-limiting examples of botulinum toxin B include rimabotulinumtoxinB and MYOBLOC®.

In embodiments that refer to a method of prevention as described herein, such embodiments are also further embodiments for use in that prevention, or alternatively for the manufacture of a medicament for use in that prevention.

The present disclosure provides a method of preventing migraine headaches in a human patient comprising administering a therapeutically effective amount of an anti-human CGRP (SEQ ID NO: 5) antibody to the human patient in need thereof; wherein the human patient was previously treated with at least one migraine preventative medication that is not an anti-human CGRP antibody or an anti-human CGRP receptor antibody. In some embodiments, the patient was previously treated with at least two different migraine preventative medications.

The present disclosure provides an anti-human CGRP (SEQ ID NO: 5) antibody for use in the prevention of migraine headaches in a human patient that was previously treated with at least one migraine preventative medication that is not an anti-human CGRP antibody or an anti-human CGRP receptor antibody.

The present disclosure provides the use of an anti-human CGRP (SEQ ID NO: 5) antibody for the manufacture of a medicament for the prevention of migraine headaches in a human patient that was previously treated with at least one migraine preventative medication that is not an anti-human CGRP antibody or an anti-human CGRP receptor antibody.

The present disclosure provides a pharmaceutical composition comprising an anti-human CGRP antibody for use in the prevention of migraine headaches in a human patient that was previously treated with at least one migraine preventative medication that is not an anti-human CGRP antibody or an anti-human CGRP receptor antibody.

The present disclosure provides a method of preventing migraine headaches in a human patient comprising administering a therapeutically effective amount of an anti-human CGRP (SEQ ID NO: 5) antibody to the human patient in need thereof, wherein the human patient was previously treated with at least one migraine preventative medication that is not an anti-human CGRP antibody or an anti-human CGRP receptor antibody. In some embodiments, the patient was previously treated with at least two different migraine preventative medications, wherein the anti-human CGRP antibody is galcanezumab.

The present disclosure provides an anti-human CGRP (SEQ ID NO: 5) antibody for use in the prevention of migraine headaches in a human patient that was previously treated with at least one migraine preventative medication that is not an anti-human CGRP antibody or an anti-human CGRP receptor antibody, wherein the anti-human CGRP antibody is galcanezumab.

The present disclosure provides the use of an anti-human CGRP (SEQ ID NO: 5) antibody for the manufacture of a medicament for the prevention of migraine headaches in a human patient that was previously treated with at least one migraine preventative medication that is not an anti-human CGRP antibody or an anti-human CGRP receptor antibody.

The present disclosure provides the use of galcanezumab for the manufacture of a medicament for the prevention of migraine headaches in a human patient that was previously treated with at least one migraine preventative medication that is not an anti-human CGRP antibody or an anti-human CGRP receptor antibody.

The present disclosure provides a pharmaceutical composition comprising an anti-human CGRP antibody for use in the prevention of migraine headaches in a human patient that was previously treated with at least one migraine preventative medication that is not an anti-human CGRP antibody or an anti-human CGRP receptor antibody.

The present disclosure provides a pharmaceutical composition comprising galcanezumab for use in the prevention of migraine headaches in a human patient that was previously treated with at least one migraine preventative medication that is not an anti-human CGRP antibody or an anti-human CGRP receptor antibody.

In some embodiments, the patient was previously treated with at least three different migraine preventative medications.

In some embodiments, the patient was previously treated with at least four different migraine preventative medications. In some embodiments, at least one of the migraine preventive medications is propranolol, metoprolol, topiramate, valproate, amitriptyline, flunarizine, candesartan, botulinum toxin A, or botulinum toxin B.

In some embodiments, the patient was previously treated with at least two, preferably at least three, more preferably at least four migraine preventive medications, each of different classes, and selected from the following classes: beta-blockers; calcium channel blockers; tricyclic anti-depressants; selective serotonin reuptake inhibitors; anticonvulsants; serotonin antagonists; magnesium salts; vitamins; and herbals.

In some embodiments, the patient was previously treated with at least two, preferably at least three, more preferably at least four migraine preventive medications, each of different classes, and selected from the following classes: beta-blockers; calcium channel blockers; tricyclic anti-depressants; selective serotonin reuptake inhibitors; anticonvulsants; and serotonin antagonists.

In some embodiments, the human patient has been diagnosed with episodic migraine prior to receiving the antibody.

In some embodiments, the human patient has been diagnosed with chronic migraine prior to receiving the antibody. In some embodiments, the human patient experiences auras with their migraine headaches.

In some embodiments, the human patient does not experience auras with their migraine headaches.

In some embodiments, the antibody administered is eptinezumab or fremanezumab.

In some embodiments, the antibody administered binds to an epitope comprising amino acid residues 8-18 (i.e., VTHRLAGLLSR) of human CGRP (as shown in SEQ ID NO: 5). In a preferred embodiment, the antibody comprises a heavy chain having a heavy chain variable region having the amino acid sequence of SEQ ID NO: 2 and a light chain having a light chain variable region having the amino acid sequence of SEQ ID NO: 1. In a preferred embodiment, the antibody comprises a heavy chain having the amino acid sequence of SEQ ID NO: 4 and a light chain having the amino acid sequence of SEQ ID NO: 3.

In some embodiments, the antibody comprising a heavy chain having a heavy chain variable region having the amino acid sequence of SEQ ID NO: 2 and a light chain having a light chain variable region having the amino acid sequence of SEQ ID NO: 1 is administered subcutaneously at a dose of about 120 mg to about 240 mg, alternatively from 120 mg to 240 mg.

In some embodiments, the antibody comprising a heavy chain having a heavy chain variable region having the amino acid sequence of SEQ ID NO: 2 and a light chain having a light chain variable region having the amino acid sequence of SEQ ID NO: 1 is administered subcutaneously at a dose of about 120 mg, alternatively 240 mg.

In some embodiments, the antibody comprising a heavy chain having a heavy chain variable region having the amino acid sequence of SEQ ID NO: 2 and a light chain having a light chain variable region having the amino acid sequence of SEQ ID NO: 1 is administered subcutaneously at a dose of about 120 mg, alternatively 240 mg.

In some embodiments, the antibody comprising a heavy chain having a heavy chain variable region having the amino acid sequence of SEQ ID NO: 2 and a light chain having a light chain variable region having the amino acid sequence of SEQ ID NO: 1 is administered to the patient as a first dose of about 240 mg and a second dose of about 120 mg, wherein the first and second dose are administered about one month apart.

In some embodiments, the antibody comprising a heavy chain having a heavy chain variable region having the amino acid sequence of SEQ ID NO: 2 and a light chain having a light chain variable region having the amino acid sequence of SEQ ID NO: 1 is administered to the patient as a first dose of 240 mg and a second dose of 120 mg, wherein the first and second dose are administered about one month apart.

In some embodiments, the antibody comprising a heavy chain having a heavy chain variable region having the amino acid sequence of SEQ ID NO: 2 and a light chain having a light chain variable region having the amino acid sequence of SEQ ID NO: 1 is administered to the patient once per month at a dose of about 120 mg to about 240 mg.

Embodiments of the present invention include the following:
a) A method of preventing migraine headaches in a human patient comprising administering a therapeutically effective amount of an anti-human CGRP antibody which binds to the CGRP peptide as shown in SEQ ID NO: 5 to the human patient in need thereof, wherein the human patient was previously treated with at least one migraine preventative medication that is not an anti-human CGRP antibody or an anti-human CGRP receptor antibody.
   a. In another embodiment, the patient was previously treated with at least two different migraine preventative medications that are neither an anti-human CGRP antibody or an anti-human CGRP receptor antibody.
   b. In another embodiment, the patient was previously treated with at least three different migraine preventative medications that are neither an anti-human CGRP antibody or an anti-human CGRP receptor antibody.
   c. In another embodiment, at least one of the migraine preventive medications is propranolol, metoprolol, topiramate, valproate, amitriptyline, flunarizine, candesartan, botulinum toxin A, or botulinum toxin B.
   d. In another embodiment, the patient was previously treated with at least four migraine preventive medications that are neither an anti-human CGRP antibody or an anti-human CGRP receptor antibody, each of different classes, and selected from the following classes: beta-blockers; calcium channel blockers; tricyclic anti-depressants; selective serotonin reuptake inhibitors; anticonvulsants; serotonin antagonists; magnesium salts; vitamins; and herbals.
   e. In another embodiment, the patient was previously treated with at least four migraine preventive medications that are neither an anti-human CGRP antibody or an anti-human CGRP receptor antibody, each of different classes, and selected from the following classes: beta-blockers; calcium channel blockers; tricyclic anti-depressants; selective serotonin reuptake inhibitors; anticonvulsants; and serotonin antagonists.
   f. In another embodiment, the human patient has been diagnosed with episodic migraine or chronic migraine prior to receiving the antibody.
   g. In another embodiment, the human patient experiences auras with their migraine headaches.
   h. In another embodiment, the human patient does not experience auras with their migraine headaches.
   i. In another embodiment, the antibody administered is eptinezumab or fremanezumab.
   j. In another embodiment, the anti-human CGRP antibody administered binds to an epitope comprising amino acid residues 8-18 (i.e., VTHRLAGLLSR) of human CGRP (as shown in SEQ ID NO: 5) as determined by hydrogen-deuterium exchange.
   k. In another embodiment, the anti-human CGRP antibody comprises a heavy chain having a heavy chain variable region having the amino acid sequence of SEQ ID NO: 2 and a light chain having a light chain variable region having the amino acid sequence of SEQ ID NO: 1.
   l. In another embodiment, the anti-human CGRP antibody comprises a heavy chain having the amino acid sequence of SEQ ID NO: 4 and a light chain having the amino acid sequence of SEQ ID NO: 3.
   m. In another embodiment, the anti-human CGRP antibody is administered subcutaneously at a dose of about 120 mg to about 240 mg. In another embodiment, the anti-human CGRP antibody is administered subcutaneously at a dose of about 120 mg.
   n. In another embodiment, the anti-human CGRP antibody is administered subcutaneously at a dose of about 240 mg.
   o. In another embodiment, the human patient is administered a first dose of about 240 mg and a second dose of about 120 mg and wherein the first and second dose are administered about one month apart.
   p. In another embodiment, the antibody is administered once per month.
b) An anti-human CGRP (SEQ ID NO: 5) antibody for use in the prevention of migraine headaches in a human patient that was previously treated with at least one, at least two different, at least three different, or at least four different migraine preventative medications that are neither an anti-human CGRP antibody or an anti-human CGRP receptor antibody.
c) An anti-human CGRP (SEQ ID NO: 5) antibody for use in the prevention of migraine headaches in a human patient that was previously treated with at least one, at least two different, at least three different, or at least four different migraine preventative medications that are neither an anti-human CGRP antibody or an anti-human CGRP receptor antibody, wherein at least one of the migraine preventive medication is propranolol, metoprolol, topiramate, valproate, amitriptyline, flunarizine, candesartan, botulinum toxin A, or botulinum toxin B.
d) An anti-human CGRP (SEQ ID NO: 5) antibody for use in the prevention of migraine headaches in a human patient that was previously treated with at least 4 migraine preventative medications that are neither an anti-human CGRP antibody or an anti-human CGRP receptor antibody, each of different classes, and selected from the group consisting of beta-blockers, calcium channel blockers, tricyclic anti-depressants, selective serotonin reuptake inhibitors, anticonvulsants, serotonin antagonists, magnesium salts, vitamins; and herbals.
e) An anti-human CGRP (SEQ ID NO: 5) antibody for use in the prevention of migraine headaches in a human patient that was previously diagnosed with episodic migraine or chronic migraine prior to receiving the antibody and previously treated with at least 4 migraine preventative medications that are neither an anti-human CGRP antibody or an anti-human CGRP receptor antibody, each of different classes, and selected from the group consisting of beta-blockers, calcium channel blockers, tricyclic anti-depressants, selective serotonin reuptake inhibitors, anticonvulsants, serotonin antagonists, magnesium salts, vitamins; and herbals, and wherein the patient either does or does not experience auras with their migraine headache.
f) An anti-human CGRP (SEQ ID NO: 5) antibody for use in the prevention of migraine headaches in a human patient that was previously treated with at least 4 migraine preventative medications that are neither an anti-human CGRP antibody or an anti-human CGRP receptor antibody, each of different classes, and selected from the group consisting of beta-blockers, calcium channel blockers, tricyclic anti-depressants, selective serotonin reuptake inhibitors, anticonvulsants, serotonin antagonists, magnesium salts, vitamins; and herbals. such an antibody is eptinezumab or fremanezumab.
g) An anti-human CGRP (SEQ ID NO: 5) antibody for use in the prevention of migraine headaches in a human patient that was previously treated with at least 4 migraine preventative medications that are neither an anti-human CGRP antibody or an anti-human CGRP receptor antibody, each of different classes, and selected from the group consisting of beta-blockers, calcium channel blockers, tricyclic anti-depressants, selective serotonin reuptake inhibitors, anticonvulsants, serotonin antagonists, magnesium salts, vitamins; and herbals, wherein such an antibody is eptinezumab or fremanezumab.
h) An anti-human CGRP (SEQ ID NO: 5) antibody for use in the prevention of migraine headaches in a human patient that was previously treated with at least 4 migraine preventative medications that are neither an anti-human CGRP antibody or an anti-human CGRP receptor antibody, each of different classes, and selected from the group consisting of beta-blockers, calcium channel blockers, tricyclic anti-depressants, selective serotonin reuptake inhibitors, anticonvulsants, serotonin antagonists, magnesium salts, vitamins; and herbals, wherein the antibody administered binds to an epitope comprising amino acid residues 8-18 (i.e., VTHRLAGLLSR) of human CGRP (as shown in SEQ ID NO: 5) as determined by hydrogen-deuterium exchange.
   In some embodiments of the embodiments b)-h) directly above, the antibody for use comprises a heavy chain having a heavy chain variable region having the amino acid sequence of SEQ ID NO: 2 and a light chain having a light chain variable region having the amino acid sequence of SEQ ID NO: 1. In other embodiments, the antibody for use comprises a heavy chain having a heavy chain variable region having the amino acid sequence of SEQ ID NO: 2 and a light chain having a light chain variable region having the amino acid sequence of SEQ ID NO: 1. In other embodiments, the antibody for use comprises a heavy chain having the amino acid sequence of SEQ ID NO: 4 and a light chain having the amino acid sequence of SEQ ID NO: 3. In other embodiments, the antibody for use is administered subcutaneously at a dose of about 120 mg to about 240 mg, about 120 mg or about 240 mg. In other embodiments, the antibody for use, is administered as a first dose of about 240 mg and a second dose of about 120 mg and wherein the first and second dose are administered about one month apart. In other embodiments, the antibody for use, is administered once per month.
i) A use of an anti-human CGRP (SEQ ID NO: 5) antibody for the manufacture of a medicament for the prevention of migraine headaches in a human patient that was previously treated with at least one migraine preventative medication that is not an anti-human CGRP or an anti-human CGRP receptor antibody.
j) A pharmaceutical composition comprising an anti-human calcitonin CGRP antibody for use in the prevention of migraine headaches in a human patient that was previously treated with at least one migraine preventative medication that is not an anti-human CGRP antibody or an anti-human CGRP receptor antibody.
k) A pharmaceutical composition comprising an anti-human calcitonin CGRP antibody for use in the prevention of migraine headaches in a human patient that was previously treated with at least two migraine preventative medication that is not an anti-human CGRP antibody or an anti-human CGRP receptor antibody.
l) A pharmaceutical composition comprising an anti-human calcitonin CGRP antibody for use in the prevention of migraine headaches in a human patient that was previously treated with at least three migraine preventative medications that are not an anti-human CGRP antibody or an anti-human CGRP receptor antibody.
   In some embodiments of the embodiments 0-1) directly above, at least one of the migraine preventive medications is propranolol, metoprolol, topiramate, valproate, amitriptyline, flunarizine, candesartan, botulinum toxin A, or botulinum toxin B. In other such embodiments, the antibody comprises a heavy chain having a heavy chain variable region having the amino acid sequence of SEQ ID NO: 2 and a light chain having a light chain variable region having the amino acid sequence of SEQ ID NO: 1. In other such embodiments, the antibody comprises a heavy chain having the amino acid sequence of SEQ ID NO: 4 and a light chain having the amino acid sequence of SEQ ID NO: 3.

The inventions disclosed herein derive from a randomized, double-blind, placebo-controlled study of galcanezumab (an anti-CGRP antibody having a heavy chain having the amino acid sequence of SEQ ID NO: 4 and a light chain having the amino acid sequence of SEQ ID NO: 3, and further described, for example, in PCT International Publication Number WO2011/156324A1) in adults with treatment-resistant migraine. This study (referred to hereinbelow as “Study CGAW”) will test the primary hypothesis that galcanezumab is superior to placebo in the prevention of migraine in patients with treatment-resistant migraine. The primary endpoint of this study is the overall mean change from baseline in the number of monthly migraine headache days during the 3-month double-blind treatment phase in the total population (episodic and chronic migraine).

All key secondary objectives will be tested in both the total population (episodic and chronic migraine) and the episodic migraine subpopulation unless otherwise specified. The specific methodology (including testing order and population) for the tests of the following key secondary endpoints will be specified in the statistical analysis plan.

A secondary objective of the study is to compare galcanezumab with placebo with respect to the prevention of migraine in the episodic migraine subpopulation where the secondary objective reflects the overall mean change from baseline in the number of monthly migraine headache days during the 3-month double-blind treatment phase in patients with episodic migraine. A secondary objective of the study is to compare galcanezumab with placebo with respect to 50% response rate where the secondary objective reflects the percentage of patients with ≥50% reduction from baseline in monthly migraine headache days during the 3-month double-blind treatment phase. A secondary objective of the study is to compare galcanezumab with placebo with respect to change in functioning where the secondary objective reflects the mean change from baseline in the Role Function-Restrictive domain score of the Migraine-Specific Quality of Life Questionnaire version 2.1 (MSQ v2.1) at Month 3. A secondary objective of the study is to compare galcanezumab with placebo with respect to 75% response rate where the secondary objective reflects the percentage of patients with ≥75% reduction from baseline in monthly migraine headache days during the 3-month double-blind treatment phase. A secondary objective of the study is to compare galcanezumab with placebo with respect to 100% response rate where the secondary objective reflects the percentage of patients with 100% reduction from baseline in monthly migraine headache days during the 3-month double-blind treatment phase.

Study CGAW is a multicenter, randomized, double-blind, parallel, placebo-controlled study of galcanezumab in patients who meet International Classification of Headache Disorders (ICHD) criteria for a diagnosis of migraine with or without aura or chronic migraine, and who have previously failed 2 to 4 standard-of-care treatments for migraine prevention. The study has 4 periods, including a prospective baseline period to determine patient eligibility.

The study has two treatment arms: galcanezumab (120 mg/month, with a 240-mg loading dose) and placebo. Following a 1-month prospective baseline period, eligible patients will be randomized in a 1:1 ratio to receive placebo or galcanezumab for up to 3 months of double-blind treatment. Investigational product is administered as 1 or 2 subcutaneous injections per month (2 injections of 120-mg galcanezumab or 2 injections of placebo at randomization; 1 injection of 120-mg galcanezumab or 1 injection of placebo at the subsequent double-blind dosing visits). Patients who complete the double-blind treatment phase may enter a 3-month open-label treatment phase. At the first dosing visit in the open-label treatment phase, patients previously assigned to placebo will receive an initial loading dose of galcanezumab 240 mg (2 injections of 120 mg each), while patients previously assigned to galcanezumab will receive 1 injection of 120-mg galcanezumab and 1 injection of placebo to retain blinding of dose assignment from the double-blind phase. Thereafter, all patients in the open-label treatment phase will receive 120 mg per month of galcanezumab.

The study will screen an estimated 764 potential study participants to ensure randomization of approximately 420 patients with migraine, of which approximately 250 patients have episodic migraine. Unless otherwise specified, statistical analyses will be conducted on an intent-to-treat (ITT) population, which includes all patients who are randomized and receive at least one dose of investigational product. Patients in the ITT population will be analyzed according to the treatment group to which they are randomized. When change from baseline is assessed, the patient will be included in the analysis only if he/she has a baseline and a post-baseline measurement. The primary analysis will evaluate the efficacy of galcanezumab compared with placebo on the overall mean change from baseline in the number of monthly migraine headache days and probable migraine headache days during the 3-month double-blind treatment phase. The primary analysis will be performed using a restricted maximum likelihood-based mixed model repeated measures technique.

Patients are eligible to be included in the study only if they meet all of the following criteria at screening: 1) are 18 to 75 years of age (inclusive) at the time of screening; 2) have a diagnosis of migraine as defined by International Headache Society ICHD-3 guidelines (1.1, 1.2, or 1.3) (ICHD-3 2018), with a history of migraine headaches of at least 1 year prior to visit 1, and migraine onset prior to age 50; 3) have a history, prior to visit 1, of at least 4 migraine headache days and at least 1 headache-free day per month on average within the past 3 months; 4) have, prior to visit 1, documentation (medical or pharmacy record or by physician's confirmation) of previous failure to 2 to 4 migraine preventive medication categories in the past 10 years from the following list due to inadequate efficacy (that is, maximum tolerated dose for at least 2 months) and/or safety/tolerability reasons, the list including (a) propranolol or metoprolol (b) topiramate, (c) valproate or divalproex, (d) amitriptyline, (e) flunarizine, (f) candesartan, (g) botulinum toxin A or B, and (h) medication locally approved for the prevention of migraine (patients only qualifying under (f) and (h) should not exceed 20% of the total study population); 5) from visit 2 to visit 3 (prospective baseline period), have a frequency of 4 or more migraine headache days and at least 1 headache-free day per 30-day period (to avoid biased reporting, patients will not be told the number of migraine or headache days on which study qualification is based); and 6) from visit 2 to visit 3 (prospective baseline period), must achieve sufficient compliance with ePRO daily headache entries as demonstrated by completion of at least 80% of daily diary entries.

Essentially as described above, galcanezumab is compared to placebo in a double-blind, phase 3 clinical study in patients with treatment-resistant migraine. The results from the 3-month double-blind treatment phase of the study revealed that galcanezumab-treated patients had a significantly greater reduction in monthly migraine headache days versus placebo. More specifically, galcanezumab-treated patients had an average of 4.1 fewer monthly migraine headache days from a baseline of 13.4, while placebo-treated patients averaged 1.0 fewer from a baseline of 13.0 (between-group difference of −3.1; p<0.0001; 95% CI −3.9 to 2.3; effect size=0.72). It is also notable that galcanezumab was superior to placebo on all key secondary endpoints. There were no statistically significant differences between galcanezumab and placebo on treatment-emergent adverse events except for those more frequently reported in the placebo group. One galcanezumab-treated patient discontinued early due to an adverse event (rash). Thus, galcanezumab was superior to placebo in the preventive treatment of treatment-resistant migraine. Additionally, galcanezumab was also safe and well-tolerated in patients who had previous failures to standard-of-care preventive treatments.

Sequences
LCVR of Galcanezumab (Artificial Sequence)
SEQ ID NO: 1
DIQMTQSPSSLSASVGDRVTITCRASKDISKYLNWYQQKPGKAPKLLIYY
TSGYHSGVPSRFSGSGSGTDFTLTISSLQPEDFATYYCQQGDALPPTFGG
GTKVEIK
HCVR of Galcanezumab (Artificial Sequence)
SEQ ID NO: 2
QVQLVQSGAEVKKPGSSVKVSCKASGYTFGNYWMQWVRQAPGQGLEWMGA
IYEGTGKTVYIQKFADRVTITADKSTSTAYMELSSLRSEDTAVYYCARLS
DYVSGFGYWGQGTTVTVSS
LC of Galcanezumab (Artificial Sequence)
SEQ ID NO: 3
DIQMTQSPSSLSASVGDRVTITCRASKDISKYLNWYQQKPGKAPKLLIYY
TSGYHSGVPSRFSGSGSGTDFTLTISSLQPEDFATYYCQQGDALPPTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
HC of Galcanezumab (Artificial Sequence)
SEQ ID NO: 4
QVQLVQSGAEVKKPGSSVKVSCKASGYTFGNYWMQWVRQAPGQGLEWMGA
IYEGTGKTVYIQKFADRVTITADKSTSTAYMELSSLRSEDTAVYYCARLS
DYVSGFGYWGQGTTVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEAAGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLG
Human αCGRP Peptide (Homo sapiens)
SEQ ID NO: 5
ACDTATCVTHRLAGLLSRSGGVVKNNFVPTNVGSKAF

1. A method of preventing migraine headaches in a human patient comprising administering a therapeutically effective amount of an anti-human calcitonin gene-related peptide (CGRP) antibody which binds to a CGRP peptide having an amino acid sequence as shown in SEQ ID NO: 5 to the human patient in need thereof; wherein the human patient was previously treated with at least three migraine preventative medications, none of which are anti-human CGRP antibodies and/or anti-human CGRP receptor antibodies.

2. The method of claim 1, wherein at least one of the migraine preventive medications is propranolol, metoprolol, topiramate, valproate, amitriptyline, flunarizine, candesartan, botulinum toxin A, or botulinum toxin B.

3. The method of claim 1, wherein the patient was previously treated with at least three migraine preventive medications that are neither an anti-human CGRP antibody or an anti-human CGRP receptor antibody, each of different classes, and selected from the following classes:
(a) beta-blockers;
(b) calcium channel blockers;
(c) tricyclic anti-depressants;
(d) selective serotonin reuptake inhibitors;
(e) anticonvulsants;
(f) serotonin antagonists;
(g) magnesium salts;
(h) vitamins; and
(i) herbals.

4. The method of claim 3, wherein the human patient has been diagnosed with episodic migraine or chronic migraine prior to receiving the antibody.

5. The method of claim 4, wherein the human patient experiences auras with their migraine headaches.

6. The method of claim 4, wherein the human patient does not experience auras with their migraine headaches.

7. The method of claim 4, wherein the antibody administered is eptinezumab or fremanezumab.

8. The method of claim 4, wherein the antihuman CGRP antibody administered to the patient comprises a heavy chain having a heavy chain variable region having the amino acid sequence of SEQ ID NO: 2 and a light chain having a light chain variable region having the amino acid sequence of SEQ ID NO: 1.

9. The method of claim 1, wherein the antibody is administered subcutaneously at a dose of about 120 mg to about 240 mg.

10-39. (canceled)

40. The method of claim 2, wherein the human patient has been diagnosed with episodic migraine or chronic migraine prior to receiving the antibody.

41. The method of claim 40, wherein the antibody is administered subcutaneously at a dose of about 120 mg to about 240 mg.

42. A method of preventing migraine headaches in a human patient comprising administering a therapeutically effective amount of an anti-human calcitonin gene-related peptide (CGRP) antibody which binds to a CGRP peptide having an amino acid sequence as shown in SEQ ID NO: 5 to the human patient in need thereof; wherein the antibody comprises a heavy chain having a heavy chain variable region having the amino acid sequence of SEQ ID NO: 2 and a light chain having a light chain variable region having the amino acid sequence of SEQ ID NO: 1, wherein the human patient has been diagnosed with episodic migraine or chronic migraine prior to receiving the antibody, and wherein the patient was previously treated with at least one migraine preventative medication that is not an anti-human CGRP antibody or an anti-human CGRP receptor antibody.

43. The method of claim 42, wherein the antibody is administered subcutaneously at a dose of about 120 mg to about 240 mg.

44. The method of claim 43, wherein at least one of the migraine preventive medications is propranolol, metoprolol, topiramate, valproate, amitriptyline, flunarizine, candesartan, botulinum toxin A, or botulinum toxin B.

45. The method of claim 43, wherein the patient was previously treated with at least three migraine preventive medications that are neither an anti-human CGRP antibody or an anti-human CGRP receptor antibody, each of different classes, and selected from the following classes:
(j) beta-blockers;
(k) calcium channel blockers;
(l) tricyclic anti-depressants;
(m) selective serotonin reuptake inhibitors;
(n) anticonvulsants;
(o) serotonin antagonists;
(p) magnesium salts;
(q) vitamins; and
(r) herbals.

46. The method of claim 44, wherein the human patient has been diagnosed with episodic migraine or chronic migraine prior to receiving the antibody.

47. The method of claim 46, wherein the human patient experiences auras with their migraine headaches.

48. The method of 47, wherein the human patient does not experience auras with their migraine headaches.

49. The method of claim 47, wherein the antibody administered is eptinezumab or fremanezumab.

50. The method of claim 48, wherein the antibody administered is eptinezumab or fremanezumab.
